(19) United States
(12) Patent Application Publication
(10) Pub. No.: US 2020/0203009 A1
(43) Pub. Date: Jun. 25, 2020

(54) ENDOSCOPE MANAGEMENT DEVICE AND METHOD OF MANAGING AN ENDOSCOPE

(71) Applicant: OLYMPUS CORPORATION, Tokyo (JP)

(72) Inventors: Haruhiko SAKAYORI, Tokyo (JP); Ryosuke SAKAI, Tokyo (JP); Kazuyuki INOKIHARA, Tokyo (JP); Yutaka SUGANUMA, Tokyo (JP)

(73) Assignee: OLYMPUS CORPORATION, Tokyo (JP)

(21) Appl. No.: 16/809,756

(22) Filed: Mar. 5, 2020

Related U.S. Application Data
(63) Continuation of application No. PCT/JP2018/020111, filed on May 25, 2018.

(30) Foreign Application Priority Data
Sep. 6, 2017 (JP) 2017-171413

(51) Int. Cl. G16H 40/40; G06F 16/23

(52) U.S. Cl. CPC G16H 40/40; G06F 16/2379

(57) ABSTRACT

An endoscope information registration unit registers information of an endoscope that is not stored in a master information storage in the master information storage. The endoscope information registration unit registers information of a second endoscope in the master information storage after a cleaning information registration unit registers cleaning information indicating that cleaning of a first endoscope has been abnormally terminated in a cleaning history storage, and a replacement determination unit determines that the second endoscope is a replacement for the first endoscope when a usage information registration unit registers usage information of the first endoscope or the cleaning information registration unit registers cleaning information of the first endoscope in the cleaning history storage thereafter.

### CROSS-REFERENCE TO RELATED APPLICATIONS

This application is based upon and claims the benefit of priority from the prior Japanese Patent Application No. 2017-171413, filed on Sep. 6, 2017 and International Application No. PCT/JP2018/020111, filed on May 25, 2018, the entire contents of which are incorporated herein by reference.

### BACKGROUND OF THE INVENTION

### 1. Field of the Invention

The present invention relates to a technology for managing endoscopes at a medical facility.

### 2. Description of the Related Art

Patent document 1 discloses a medical service support system in which a management facility system and a medical facility system are connected via a network. In the medical service support system disclosed in Patent document 1, the management facility system registers the status of a faulty instrument transported from a medical facility to a management facility and the status of a replacement instrument transported from the management facility to the medical facility and gives notification of status information that is registered to the medical facility system. Based on the status information as notified, the medical facility system registers an unavailable status of the faulty instrument and an unavailable status of the replacement instrument, and when the medical facility system determines that the replacement instrument is available, the medical facility system changes the unavailable status of the replacement instrument to an available status.

[Patent document 1] Japanese Patent Application Publication No. 2013-228946

When an endoscope fails, a medical facility sends the failed endoscope to the manufacturer and requests for repair. During the repair, the medical facility registers a replacement provided by the manufacturer in an endoscope master table and uses the replacement in place of the failed endoscope. When the manufacturer completes the repair, the medical facility receives the endoscope that has been repaired and returns the replacement to the manufacturer. At this time, if information of the replacement remains valid in the master table, that is, if the information remains in a state accessible to the user, erroneous data entry may be caused. Therefore, information of the replacement that has been returned is preferably invalidated (including deletion) immediately in the master table. For that purpose, it is necessary to identify which endoscope information in the master table is the information of the replacement. However, a replacement identification process is preferably performed easily without causing trouble to the user as much as possible.

### SUMMARY OF THE INVENTION

In this background, a purpose of the present invention is to provide a technology for managing endoscopes in a facility.

An endoscope management device according to one embodiment of the present invention includes: a master information storage that stores information of an endoscope; an endoscope information registration unit that registers information of the endoscope that is not stored in the master information storage in the master information storage; a usage history storage that stores usage information of an endoscope used in an examination; a usage information registration unit that registers usage information of the endoscope in the usage history storage; a cleaning history storage that stores cleaning information on cleaning of the endoscope; a cleaning information registration unit that registers cleaning information of the endoscope in the cleaning history storage; and a replacement determination unit that determines that a second endoscope is a replacement for a first endoscope based on cleaning information of the first endoscope stored in the cleaning history storage.

Optional combinations of the aforementioned constituting elements and implementations of the invention in the form of methods, apparatuses, systems, recording mediums, and computer programs may also be practiced as additional modes of the present invention.

### BRIEF DESCRIPTION OF THE FIGURE

Embodiments will now be described, by way of example only, with reference to the accompanying drawings that are meant to be exemplary, not limiting, and wherein like elements are numbered alike in several figures, in which:

FIG. 1 is a diagram showing the configuration of an endoscopic examination support system according to an embodiment;

FIG. 2 is a diagram showing an example of information stored in a master information storage;

FIG. 3 is a diagram showing an example of usage information stored in a usage history storage;

FIG. 4 is a diagram showing an example of cleaning information stored in a cleaning history storage;

FIG. 5 is a diagram showing an example of cleaning information stored in the cleaning history storage;

FIG. 6 is a diagram showing an example of information stored in the master information storage;

FIG. 7 is a diagram showing an example of usage information stored in the usage history storage;

FIG. 8 is a diagram showing an example of cleaning information stored in the cleaning history storage;

FIG. 9 is a diagram showing an example of usage information stored in the usage history storage;

FIG. 10 is a diagram showing an example of information stored in the master information storage;

FIG. 11 is a diagram showing an example of an endoscope information entry screen; and

FIG. 12 is a diagram showing an example of information stored in the master information storage.

### DETAILED DESCRIPTION OF THE INVENTION

The invention will now be described by reference to the preferred embodiments. This does not intend to limit the scope of the present invention, but to exemplify the invention.

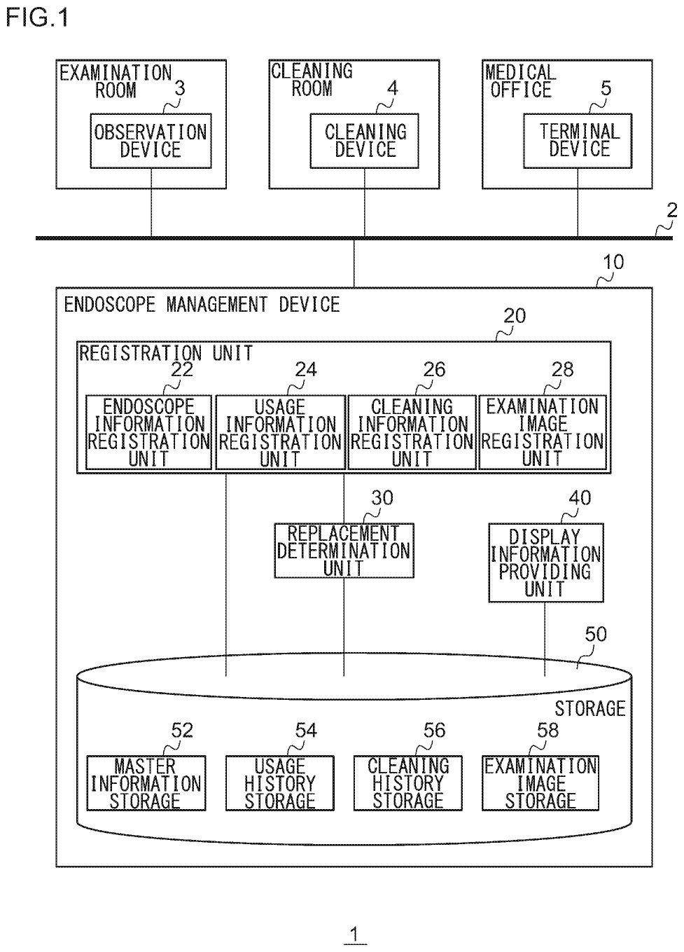

FIG. 1 shows the configuration of an endoscopic examination support system 1 according to an embodiment. In an endoscopic examination support system 1 provided in a medical facility such as a hospital, an endoscope management device 10 is communicably connected to an observation device 3 provided in an examination room, a cleaning device 4 provided in a cleaning room, and a terminal device 5 provided in a medical office via a network 2 such as a local area network (LAN).

During an examination, the observation device 3 displays image data acquired by an endoscope on a monitor in real time, acquires an examination image at the time when a release switch of the endoscope is pressed by the doctor, and transmits examination image data to the endoscope management device 10.

The cleaning device 4 automatically cleans the endoscope that is set in a cleaning tank. In addition to the automatic cleaning function, the cleaning device 4 has a failure detection function of detecting a failure of the endoscope before or during the cleaning. When the cleaning device 4 detects a failure of the endoscope, the cleaning device 4 forcibly terminates the cleaning. In the embodiment, the forced termination of cleaning due to failure detection is called “abnormal termination”. At this time, the cleaning device 4 outputs an error code indicating the cause of the abnormal termination such that the user can recognize the cause of the abnormal termination.

The terminal device 5 is an information processing device such as a personal computer or a tablet and is used when a doctor creates an examination report. The examination report includes a column for recording an endoscope used for an examination. The terminal device 5 displays an endoscope list provided from the endoscope management device 10 on a display, and the doctor selects a used endoscope from the endoscope list and records the used endoscope in the examination report.

When an endoscope owned by a medical facility breaks down, the user sends the failed endoscope to the manufacturer and requests repair, and the manufacturer provides the medical facility with an endoscope that can be used as a replacement. The endoscope provided as a replacement is usually an endoscope of the same model as the failed endoscope, but may be of a different model. When an endoscope of a different model is to be provided, the replacement needs to be of a model that can be used for an endoscopic examination for which the failed endoscope was used.

At this time, although the user ideally enters the status of the failed endoscope and the status of the endoscope received as the replacement to the endoscope management device 10 and appropriately manage the status of each endoscope, since a status change entry operation due to a failure is rare, the operation may be forgotten. In a medical facility that does not manage the status of an endoscope, there is no status change entry operation in the first place. Therefore, in the embodiment, a mechanism will be described in which the endoscope management device 10 manages endoscopes without the user entering a status change.

The endoscope management device 10 includes a registration unit 20, a replacement determination unit 30, a display information providing unit 40, and a storage 50. The registration unit 20 has an endoscope information registration unit 22, a usage information registration unit 24, a cleaning information registration unit 26, and an examination image registration unit 28. The storage 50 has a master information storage 52, a usage history storage 54, a cleaning history storage 56, and an examination image storage 58. The configuration thereof is implemented by hardware such as an arbitrary processor, a memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

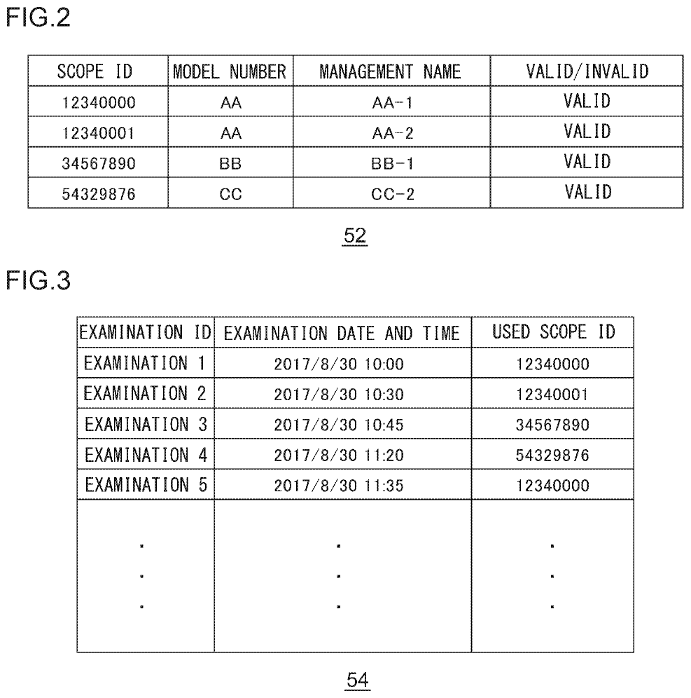

FIG. 2 shows an example of information stored in the master information storage 52. The master information storage 52 is a master table of endoscopes in a medical facility and stores information of the endoscopes. “Scope ID” is information for identifying an endoscope and may be a serial number. “Model number” is information for identifying the type (model) of an endoscope. The scope ID and model number information are set for each endoscope by the manufacturer. “Management name” is a common name of an endoscope and is added to make it easy to distinguish individual endoscopes in a medical facility. A management name is created by a combination of a model number and a number in this case. However, the management name may be created freely. “Valid/Invalid” is information indicating whether the use of endoscope information is valid or invalid and may be set as a flag value. “Valid” means that the use of the endoscope information is valid, that is, the endoscope information can be used. “Invalid” means that the use of the endoscope information is invalid, that is, the endoscope information cannot be used. The endoscope information for which an “invalid” flag is set may be invalidated by being deleted from the master information storage 52.

The endoscope information registration unit 22 registers information of the endoscope that is not stored in the master information storage 52 in the master information storage 52. In the endoscopic examination support system 1, when an endoscope is connected to the observation device 3 and is cleaned in the cleaning device 4, each of the observation device 3 and the cleaning device 4 transmits the scope ID and the model number information of the endoscope to the endoscope management device 10. If the transmitted scope ID is not stored in the master information storage 52, the endoscope information registration unit 22 registers the scope ID and the model number information in the master information storage 52 at this time. At this time, the endoscope information registration unit 22 registers a “valid” flag in a “valid/invalid” column.

FIG. 3 shows an example of usage information stored in the usage history storage 54. The usage history storage 54 stores usage information of an endoscope used in the examination. “Examination ID” is information for identifying an examination, “examination date and time” is the start date and time or the end date and time of the examination, and “used scope ID” is the scope ID of an endoscope used in the examination. When a plurality of endoscopes are used in a single examination, a plurality of used scope IDs are stored in association with the examination ID.

Before the start of an examination, when the endoscope is connected to the observation device 3 by a nurse, the observation device 3 acquires the scope ID and model number information of the endoscope. When an examination start button provided on the observation device 3 is operated, the observation device 3 transmits information indicating the start of the examination to the endoscope management device 10 along with the scope ID and the model number information so as to start the endoscopic examination. During an examination, the observation device 3 displays image data acquired by the endoscope on a monitor in real time, acquires an examination image at the time when a release switch of the endoscope is pressed, and transmits the examination image to the endoscope management device 10. The examination image registration unit 28 acquires examination image data and stores the examination image data in the examination image storage 58. When an examination end button provided on the observation device 3 is operated, the observation device 3 transmits information indicating that the examination has been ended to the endoscope management device 10, and the endoscope examination is ended.

The usage information registration unit 24 registers the usage information of the endoscope in the examination in the usage history storage 54. Although the “examination date and time” may be either the start date and time or the end date and time of the examination, both the start date and time and the end date and time may be stored. Although FIG. 3 only shows usage information for one day, the usage information registration unit 24 registers the usage information of the endoscope in a daily examination in the usage history storage 54.

As described above, when the scope ID transmitted from the observation device 3 is not stored in the master information storage 52, the endoscope information registration unit 22 registers the scope ID and the model number information in the master information storage 52 as new information of the endoscope.

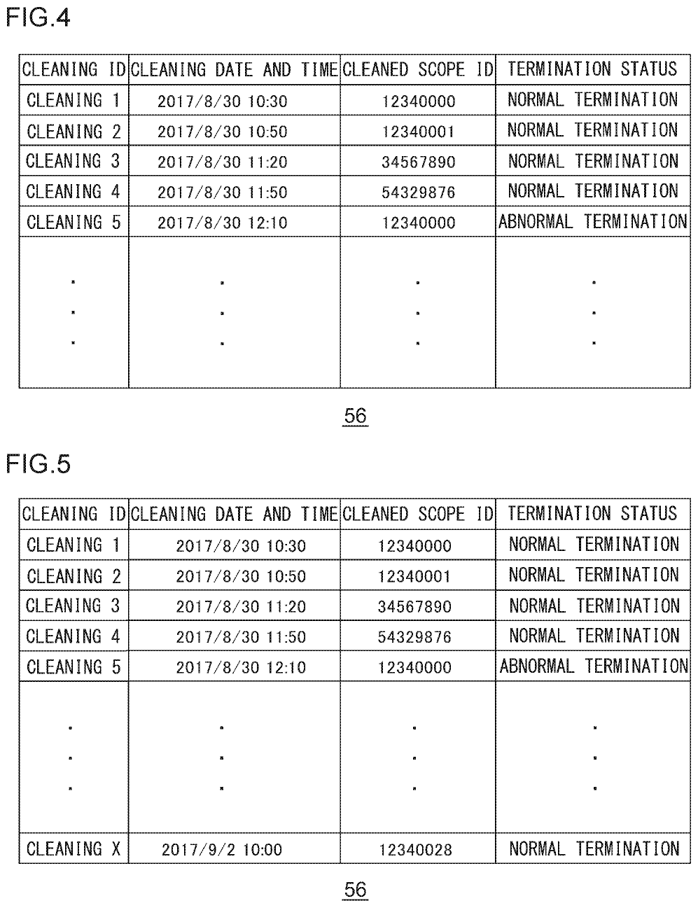

FIG. 4 shows an example of cleaning information stored in the cleaning history storage 56. The cleaning history storage 56 stores cleaning information on cleaning of endoscopes. “Cleaning ID” is information for identifying cleaning, “cleaning date and time” is the start date and time or the end date and time of the cleaning, and “cleaned scope ID” is the scope ID of a cleaned endoscope. “Termination status” is information indicating whether or not cleaning has been terminated normally, “normal termination” indicates that cleaning has been terminated normally, and “abnormal termination” indicates that a failure of an endoscope has been detected, forcibly terminating cleaning.

Before the start of cleaning, a person in charge of the cleaning brings a tape, on which the scope ID and the model number information of the endoscope are printed, into contact with an ID reader (not shown) arranged in the cleaning device 4 or holds the tape over the ID reader such that the scope ID and the model number information are read. After causing the ID reader to read the scope ID and the model number information, the person in charge of the cleaning sets the endoscope in a cleaning tank of the cleaning device 4, selects a cleaning program of the endoscope, and then operates a cleaning start button so as to start the cleaning. When the cleaning start button is operated, the cleaning device 4 transmits the scope ID and the model number information read by the ID reader to the endoscope management device 10 along with cleaning start information. When the cleaning is terminated, the cleaning device 4 automatically transmits cleaning end information to the endoscope management device 10. At this time, the cleaning device 4 transmits to the endoscope management device 10 “normal termination” information if the cleaning has been terminated normally and “abnormal termination” information if the cleaning has been forcibly terminated due to the detection of an endoscope failure.

The cleaning information registration unit 26 registers cleaning information on cleaning of an endoscope in the cleaning history storage 56. Although the “cleaning date and time” may be either the start date and time or the end date and time of the cleaning, both the start date and time and the end date and time may be stored. Although FIG. 4 only shows cleaning information for one day, the cleaning information registration unit 26 registers daily cleaning information in the cleaning history storage 56.

As described above, when the scope ID transmitted from the cleaning device 4 is not stored in the master information storage 52, the endoscope information registration unit 22 registers the scope ID and the model number information in the master information storage 52 as new information of the endoscope.

When the cleaning is abnormally terminated, the cleaning device 4 transmits an error code indicating the cause of the abnormal termination to the endoscope management device 10. Therefore, the cleaning information registration unit 26 registers the error code as cleaning information of the cleaning that has been abnormally terminated. The person in charge of the cleaning is preferably notified of the error code by various means so that the person in charge of the cleaning can recognize the endoscope failure.

For example, in a case where the cleaning device 4 has a water leakage detection function, when water leakage of an endoscope is detected, the cleaning process is forcibly terminated, and an error code indicating that the water leakage is detected is output to the endoscope management device 10. In a case where the cleaning device 4 has a display panel, the detection of water leakage may be displayed on the display panel. Further, the endoscope management device 10 may notify a terminal device possessed by the person in charge of the cleaning that water leakage has been detected in an endoscope being cleaned and that the cleaning process has been forcibly terminated.

An endoscope for which water leakage has been detected has a hole and therefore needs to be sent to the manufacturer for repair. When notified that water leakage has been detected, the person in charge of the cleaning removes, from the cleaning tank, an endoscope for which cleaning has been forcibly terminated and performs manual cleaning. The endoscope that has been manually cleaned is sent to the manufacturer, and an endoscope that can be used as a replacement is provided from the manufacturer. Hereinafter, the failed endoscope is referred to as a “first endoscope”, and the endoscope provided as the replacement is referred to as a “second endoscope”. FIG. 4 shows that the cleaning of an endoscope having a scope ID of “12340000” has been abnormally terminated in a cleaning process with a cleaning ID of “cleaning 5”, and this endoscope is referred to as the “first endoscope” in the embodiment.

After the first endoscope is sent to the manufacturer, the second endoscope is provided from the manufacturer to the medical facility. When the second endoscope is connected to the observation device 3 or the scope ID or the like is read by the ID reader of the cleaning device 4, the endoscope information registration unit 22 registers the scope ID and model number information of the second endoscope in the master information storage 52.

FIG. 5 shows an example of cleaning information stored in the cleaning history storage 56. The cleaning history storage 56 shown in FIG. 5 stores information indicating that the cleaning of an endoscope with a scope ID of “12340028” has been normally terminated in a cleaning process with a cleaning ID of “cleaning X”. At this time, since the scope ID of “12340028” is not stored in the master information storage 52, the endoscope information registration unit 22 registers the scope ID of “12340028” and the model number information of “AA” as new information of the endoscope in the master information storage 52. The endoscope with the scope ID “12340028” is the “second endoscope” in the embodiment.

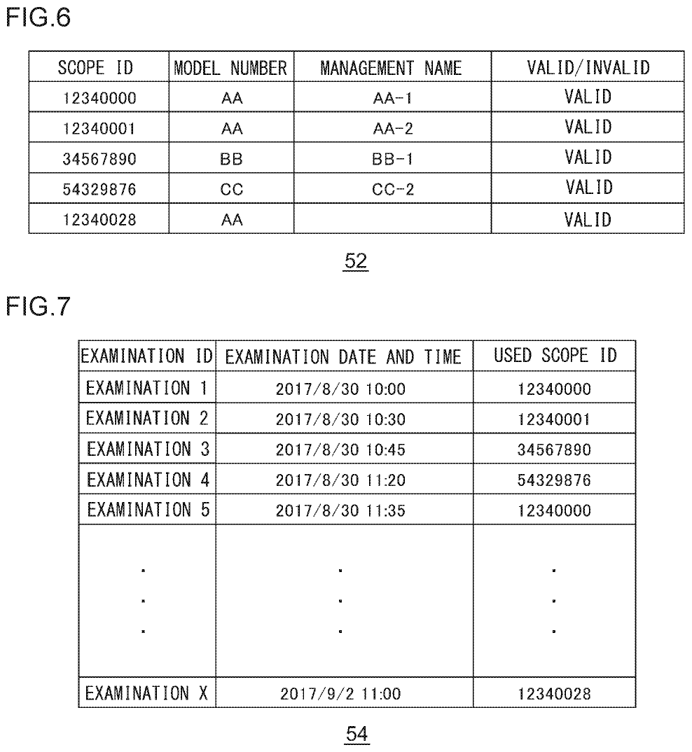

FIG. 6 shows an example of information stored in the master information storage 52. As compared with FIG. 2, it is shown that an endoscope with a scope ID of “12340028” is additionally registered in the master table. The management name is information that is arbitrarily registered on the medical facility side. When information on the second endoscope is automatically registered by the endoscope information registration unit 22, an item for the management name is not registered. The user of the medical facility can register the management name after the information on the second endoscope is automatically registered.

Shown here is a case where the second endoscope is provided to the medical facility from the manufacturer and cleaned once by the cleaning device 4 before the second endoscope is used for an examination. Since the second endoscope is provided to the facility in an already-cleaned condition, the second endoscope may be used immediately for an examination.

FIG. 7 shows an example of usage information stored in the usage history storage 54. The usage history storage 54 shown in FIG. 7 stores information indicating that an endoscope with the scope ID of “12340028” was used in an endoscopic examination with an examination ID of “examination X”. If the scope ID of “12340028” is not stored in the master information storage 52, the endoscope information registration unit 22 registers the scope ID of “12340028” and the model number information of “AA” as the new information of the endoscope in the master information storage 52 (see FIG. 6). As described above, the endoscope with the scope ID “12340028” is the “second endoscope” in the embodiment.

As described, the information of the second endoscope, which is a replacement for the first endoscope, is stored in the master information storage 52. When registering a new endoscope in the master information storage 52, the endoscope information registration unit 22 sets a “valid” flag in a “valid/invalid” column. The “valid” flag indicates that the endoscope information stored in the master information storage 52 can be used.

After the examination, the doctor operates the terminal device 5 so as to create an examination report. The doctor needs to record the endoscope used in the examination in the examination report. Therefore, the terminal device 5 requests the display information providing unit 40 to transmit the endoscope list. In response to this request, the display information providing unit 40 generates a list of endoscopes for which a valid flag is set in the master information storage 52, and transmits the list to the terminal device 5. Since a valid flag is set for the second endoscope, the information of the second endoscope is included in the list at this time. The terminal device 5 displays the list of endoscopes on the screen, and the doctor selects the endoscope that has been used and registers the endoscope in the examination report.

It usually takes several weeks to several months to repair the first endoscope. When the repair of the first endoscope is completed, the manufacturer sends the first endoscope to the medical facility, and the medical facility returns the second endoscope to the manufacturer immediately after receiving the first endoscope. In the medical facility, the first endoscope is once cleaned by the cleaning device 4 before the first endoscope is used in an examination or is connected to the observation device 3 for immediate use in the examination.

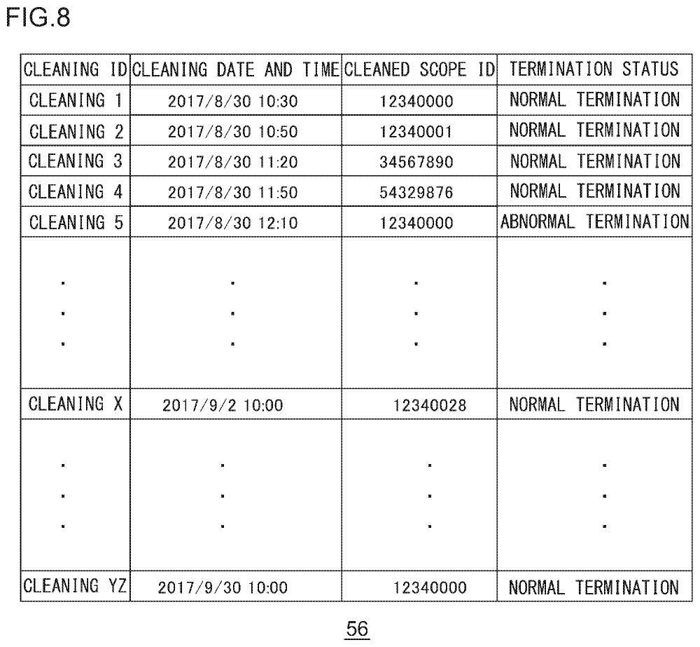

FIG. 8 shows an example of cleaning information stored in the cleaning history storage 56. The cleaning history storage 56 shown in FIG. 8 stores information indicating that the cleaning of an endoscope with a scope ID of “12340000” has been normally terminated in a cleaning process with a cleaning ID of “cleaning YZ”. The endoscope with the scope ID “12340000” is the “first endoscope” in the embodiment.

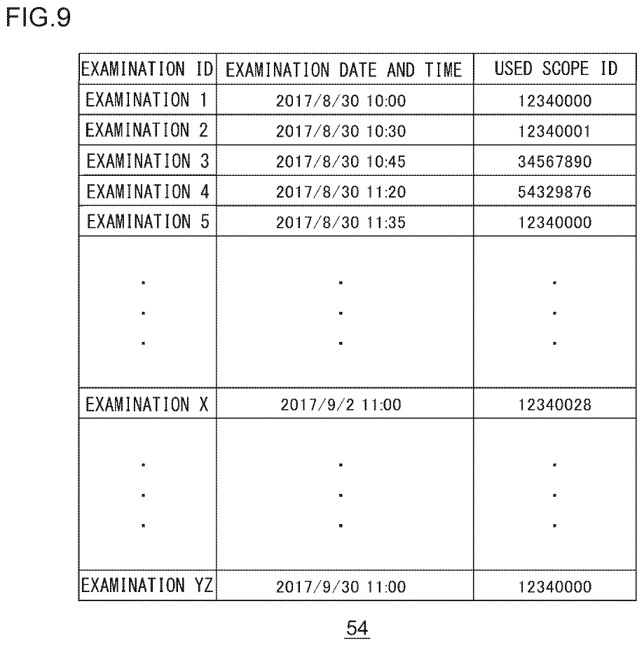

FIG. 9 shows an example of usage information stored in the usage history storage 54. The usage history storage 54 shown in FIG. 9 stores information indicating that the endoscope with the scope ID of “12340000” was used in an endoscopic examination with an examination ID of “examination YZ”. As described above, the endoscope with the scope ID “12340000” is the “first endoscope” in the embodiment.

The replacement determination unit 30 has a function of determining that the second endoscope is a replacement for the first endoscope based on at least the cleaning information of the first endoscope stored in the cleaning history storage 56. When the replacement determination unit 30 determines, at the time when the first endoscope is used by the observation device 3 or cleaned by the cleaning device 4, that the previous cleaning process of the first endoscope was abnormally terminated, the replacement determination unit 30 performs a process of identifying the second endoscope, which is the replacement, in response to the determination. More specifically, the replacement determination unit 30 determines that the second endoscope is a replacement for the first endoscope when the following conditions are satisfied.

(Condition a)

The cleaning information registration unit 26 has registered cleaning information indicating that the cleaning of the first endoscope has been abnormally terminated in the cleaning history storage 56.

(Condition b)

After the abnormal termination of the cleaning of the first endoscope and before the use or cleaning of the first endoscope, the endoscope information registration unit 22 has registered the information of the second endoscope in the master information storage 52.

(Condition c)

After the registration of the information of the second endoscope, the usage information registration unit 24 has registered the usage information of the first endoscope in the usage history storage 54, or the cleaning information registration unit 26 has registered the cleaning information of the first endoscope in the cleaning history storage 56. When these conditions a, b, and c are satisfied, the replacement determination unit 30 determines that the second endoscope is the replacement for the first endoscope.

As described above, in the embodiment, the replacement determination unit 30 determines that the second endoscope is the replacement for the first endoscope based on the conditions a to c being satisfied, without troubling the user. When it is determined that the second endoscope is the replacement, the endoscope information registration unit 22 invalidates the information of the second endoscope stored in the master information storage 52. The replacement determination unit 30 preferably determines that the second endoscope is the replacement for the first endoscope on condition that the second endoscope is of a model that can be used for an endoscopic examination in which the first endoscope was used.

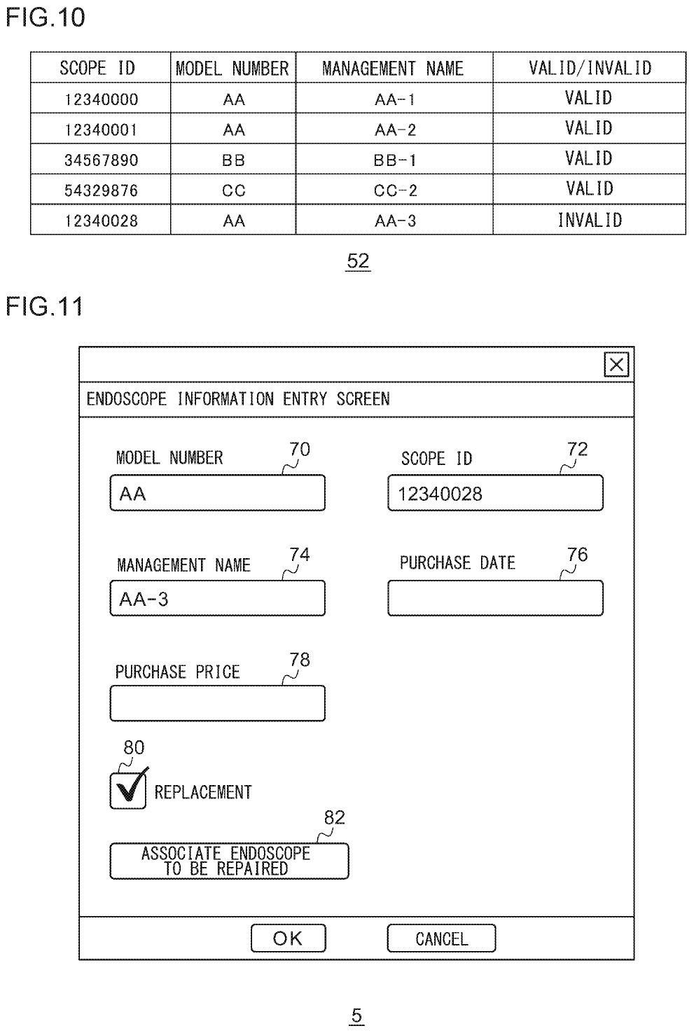

FIG. 10 shows an example of information stored in the master information storage 52. Compared to FIG. 6, an invalid flag is set in the “valid/invalid” column of the endoscope (second endoscope) with the scope ID of “12340028”. The “invalid” flag indicates that the endoscope information stored in the master information storage 52 cannot be used. A management name of “AA-3” is registered for the second endoscope. This is entered by the user in order to distinguish the second endoscope from other endoscopes in the medical facility.

At the time of report creation by the doctor, the display information providing unit 40 generates a list of endoscopes for which a valid flag is set in the master information storage 52 and transmits the list to the terminal device 5. Since an invalid flag is set for the second endoscope, the information of the second endoscope is not included in the list. Since the second endoscope has already been returned to the manufacturer, not including the information of the second endoscope in the list prevents the doctor from mistakenly selecting the second endoscope. In the embodiment, although the endoscope information registration unit 22 invalidates the information of the second endoscope by setting an invalid flag, the information of the second endoscope may be invalidated by deleting the information of the second endoscope from the master information storage 52.

Described above is an explanation on the present invention based on the embodiments. These embodiments are intended to be illustrative only, and it will be obvious to those skilled in the art that various modifications to constituting elements and processes could be developed and that such modifications are also within the scope of the present invention.

In the embodiment, a process of referring to the past history information and identifying the second endoscope as the replacement when the repaired first endoscope returns to the medical facility and is used in the examination or cleaned is shown. In an exemplary variation, a user interface for associating the second endoscope with the first endoscope is provided on an endoscope information entry screen for inputting a management name and the like of the second endoscope.

FIG. 11 shows an example of an endoscope information entry screen. When the endoscope information registration unit 22 automatically registers the second endoscope, which is the replacement, in the master information storage 52, the user operates the terminal device 5 so as to display an endoscope information entry screen for entering information regarding the second endoscope. At this time, “AA”, which is the model number of the second endoscope, is automatically entered into a model number information column 70, and the scope ID “12340028” of the second endoscope is automatically entered into a scope ID column 72. The user cannot change these pieces of information.

In a management name column 74, the user enters a name that is distinguishable from other endoscopes in the medical facility. A name “AA-3” is entered here so as to be distinguishable from the endoscope with the model number “AA” shown in FIG. 2. As a result, the second endoscope can be used under the name of “AA-3” until the first endoscope returns in the medical facility.

The purchase date of an endoscope is entered in a purchase date column 76, and the purchase price is entered in a purchase price column 78. In the exemplary variation, since the information of the second endoscope, which is the replacement, is entered, the entry into the purchase date column 76 and the purchase price column 78 is not performed.

A replacement check box 80 is provided to specify whether or not the endoscope is a replacement. When the replacement check box 80 is checked, the endoscope is registered as a replacement.

When the replacement check box 80 is checked, the operation of an association button 82 is enabled. When the association button 82 is pressed, a list of endoscopes for which a valid flag is set in the master information storage 52 is displayed. The endoscope list includes the endoscopes shown in FIG. 2. The user selects an endoscope (the first endoscope) with a scope ID “12340000”, thereby associating the first endoscope and the second endoscope.

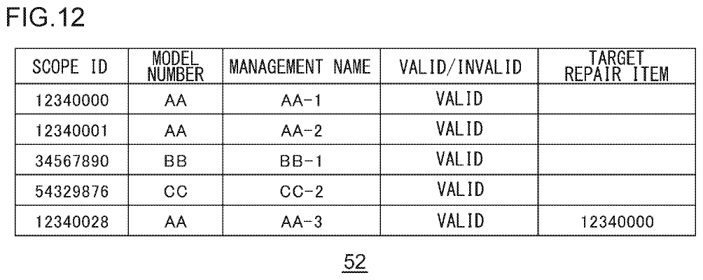

The endoscope information registration unit 22 registers the information of the second endoscope in association with the information of the first endoscope. At this time, the endoscope information registration unit 22 may set the status of the first endoscope to be a repair status. FIG. 12 shows an example of information stored in the master information storage 52. The master information storage 52 stores the information of the second endoscope in association with the scope ID of the first endoscope, which is the target repair item. As a result, when the first endoscope is returned from the manufacturer and usedin an examination or cleaned, the replacement determination unit 30 can immediately determine that the replacement for the first endoscope is the endoscope with the scope ID “12340028”.

What is claimed is:

1. An endoscope management device comprising: a master information storage that stores information of an endoscope; a usage history storage that stores usage information of an endoscope used in an examination; and a cleaning history storage that stores cleaning information on cleaning of an endoscope, and comprising: a processor comprising hardware,
wherein the processor includes functions of:
registering information of an endoscope that is not stored in the master information storage in the master information storage;
registering usage information of the endoscope in the usage history storage;
registering cleaning information of the endoscope in the cleaning history storage; and
determining that a second endoscope is a replacement for a first endoscope based on cleaning information of the first endoscope stored in the cleaning history storage, and
wherein the processor is configured to:
newly register information of the second endoscope in the master information storage after registering cleaning information indicating that cleaning of the first endoscope has been abnormally terminated in the cleaning history storage; and subsequently determine that the second endoscope is a replacement for the first endoscope upon registering usage information of the first endoscope in the usage history storage or registering cleaning information of the first endoscope in the cleaning history storage.

2. The endoscope management device according to claim 1,
wherein the processor is configured to determine that the second endoscope is the replacement for the first endoscope on condition that the second endoscope is of a model that can be used for an endoscopic examination in which the first endoscope was used.

3. The endoscope management device according to claim 1,
wherein the processor is configured to invalidate the information of the second endoscope stored in the master information storage when determining that the second endoscope is the replacement.

4. The endoscope management device according to claim 1,
wherein the cleaning information includes cleaning date and time and identification information of the endoscope.

5. The endoscope management device according to claim 1,
wherein the processor is configured to set flag information indicating that endoscope information can be used, when registering information of the second endoscope, which is the replacement for the first endoscope, in the master information storage.

6. A method of managing an endoscope in an endoscope management device comprising: a master information storage that stores information of an endoscope; a usage history storage that stores usage information of an endoscope used in an examination; and a cleaning history storage that stores cleaning information on cleaning of an endoscope, comprising:
registering cleaning information indicating that cleaning of a first endoscope has been abnormally terminated in the cleaning history storage;
newly registering information of a second endoscope in the master information storage thereafter, and
subsequently determining that the second endoscope is a replacement for the first endoscope upon registering usage information of the first endoscope in the usage history storage or registering cleaning information of the first endoscope in the cleaning history storage.
